(19) United States
(12) Patent Application Publication
(10) Pub. No.: US 2009/0299262 A1
(43) Pub. Date: Dec. 3, 2009

(54) Microporator for Creating a Permeation Surface

(75) Inventors: Thomas Bragagna, Feldkirch (AT); Reinhard Braun, Lustenau (AT); Daniel Gfrerer, Bludenz (AT); Bernhard Nussbaumer, Feldkirch (AT)

Correspondence Address: FISH & ASSOCIATES, PC;ROBERT D. FISH, 2603 Main Street, Suite 1000, Irvine, CA, 92614-6232, US

(73) Assignee: PANTEC BIOSOLUTIONS AG, Ruggell (LI)

(21) Appl. No.: 11/911,855

(22) PCT Filed: Apr. 18, 2005
(86) PCT No.: PCT/EP05/51703

§ 371 Date: Jun. 30, 2008

(51) Int. Cl. A61M 37/00; A61B 18/20

(52) U.S. Cl. 604/20; 606/9

(57) ABSTRACT

There is disclosed a method for creating an initial permeation surface (A) in a biological membrane (1) comprising: a) creating a plurality of individual micropores (2i) in the biological membrane (1), each individual micropore (2i) having an individual permeation surface (Ai); and b) creating such a number of individual micropores (2i) and of such shapes, that the initial permeation surface (A), which is the sum of the individual permeation surfaces (Ai) of all individual micropores (2i), having a desired value. A Microporator performing the method is also disclosed.

### FIELD OF THE INVENTION

This invention relates generally to the field of microporating biological membranes. More particularly, this invention relates to a method for creating an initial permeation surface in a biological membrane.

### BACKGROUND OF THE INVENTION

Many new drugs, including vaccines, proteins, peptides and DNA constituents, have been developed for better and more efficient treatment for disease, illness and cosmetic issues. However, one significant limitation in using these new substances is often a lack of an efficient drug delivery system, especially where the drug needs to be transported across one or more biological barriers at effective rates and amounts.

Transmembrane delivery can be employed which usually relies on passive diffusion of a permeant like a drug across a biological membrane such as the skin. However, transmembrane, in particular transdermal delivery is often not broadly applicable as the skin presents a relatively effective barrier for numerous drugs.

Some attempts have been made to improve transdermal delivery using a laser for puncturing the skin of a patient in a manner that does not result in bleeding. Such perforation typically penetrates through the stratum corneum or both the stratum corneum and the epidermis. This allows drug delivery through the skin. An example of such a laser, described in EP 1133953, provides one slit-shaped perforation with a width of up to 0.5 mm and a length of up to 2.5 mm. (This and all other citations herein are incorporated by reference in their entirety). Unfortunately, the rate of drug delivery through such a perforation is limited. This perforation also provokes undesirable skin reactions and the perforation of the skin frequently causes pain. The perforation requires subsequent patch drug application. However, such administration of drugs often results in inconsistent drug dosages, inconvenient usage, and sometimes even in infections.

Therefore, although there are various methods and devices for drug administration known in the art, all or almost all of them suffer from one or more disadvantages. Among other things, currently known methods and devices fail to allow controlled and reproducible administration of drugs. Currently known methods and devices also fail to provide prompt initiation and cut-off of drug delivery with improved safety, efficiency and convenience. It is therefore an object of the present invention to provide methods for creating a permeation surface in biological tissue. This problem is solved with a method for creating an initial permeation surface comprising the features of claim 1. Dependent claims 2 to 21 disclose optional methods. The problem is further solved with a method for administering a cosmetic substance comprising the features of claim 23, with dependent claims 24 disclosing optional features. The permeation surface, if used in combination with a drug, can improve transmembrane delivery of molecules, including drugs and biological molecules, across biological membranes, such as tissue or cell membranes. The permeation surface, if used in combination with a cosmetic substance, can improve intradermal delivery of the substance, to improve the cosmetic effect. The permeation surface can also be useful as such, for example, to activate cell growth for cosmetic purposes.

### SUMMARY OF THE INVENTION

The method according to the invention utilize a micro-porator for porating a biological membrane like the skin, to create a microporation consisting of a plurality of individual pores with predetermined shape. In a preferred embodiment a laser micro-porator is used. The micro-porator ablates or punctures the biological membrane, in particular the stratum corneum and part of the epidermis of the skin. This affects individual micropores in the skin, which results in an increase in skin permeability to various substances, which allows a transdermal or intradermal delivery of substances applied onto the skin. A microporation created by the microporator in one session comprises a plurality of individual pores, having a total number in the range between 10 and 1 million individual pores. By each individual pore a permeation surface within the skin is created. Depending on the number and shape of the individual pores an initial permeation surface is created, which is the sum of the permeation surfaces of all individual pores. Due to cell growth, the permeation surface of each individual pore decreases over time. The decrease of the permeation surface over time depends in particular on the geometrical shape of the individual pore. By an appropriate choice of the number of individual pores and their shape, not only the initial permeation surface but also the decrease of the permeation surface over time can be determined. The appropriate choice of number and shape can be calculated and stored as an initial microporation dataset. The micro-porator necessary for the method according to the invention has the ability to reproducibly create a microporation with a predetermined initial permeation surface and preferably also with a predetermined function of the permeation surface over time. Any biological tissue, but in particular the skin can be porated with the method according to the invention. Various techniques can be used for creating pores in biological tissues. For example also a device for heating via conductive materials or a device generating high voltage electrical pulses can be used for creating pores. U.S. Pat. No. 6,148,232, for example, disclose a technique for creating micro-channels by using an electrical field. This device could also be suitable for creating micropores of predetermined shape, if provided with means to reproducibly create micropores such as feedback means according to the invention, to detect characteristics of the individual micropores.

The amount of substances delivered through the biological membrane, in particular from the surface of the skin to within the human body, depends on the permeation surface and its variation over time. After the microporation is created, a permeant is applied onto the skin, and the transdermal or intradermal delivery of the permeant takes place depending also on the size of the permeation surface. To apply the permeant effectively, it is important to fit properties of the permeant and the microporation accordingly, to ensure a desired local or systemic effect, for example to ensure a predetermined concentration of a cosmetic substance within the skin.

As used herein, “poration” and “microporation” means the formation of a small hole or pore to a desired depth in or through the biological membrane or tissue, such as the skin, the mucous membrane or an organ of a human being or a mammal, or the outer layer of an organism or a plant, to lessen the barrier properties of this biological membrane to the passage of permeants or drugs into the body or to activate cell growth in the tissue. The microporation referred to herein shall be no smaller than 1 micron across and at least 1 micron in depth.

As used herein, “micropore”, “pore” or “individual pore” means an opening formed by the microporation method.

As used herein “ablation” means the controlled removal of material which may include cells or other components comprising some portion of a biological membrane or tissue. The ablation can be caused, for example, by one of the following:
kinetic energy released when some or all of the vaporizable components of such material have been heated to the point that vaporization occurs and the resulting rapid expansion of volume due to this phase change causes this material, and possibly some adjacent material, to be removed from the ablation site (e.g. laser, microwave, alpha-, beta- or gamma radiation, hot material);
Thermal or mechanical decomposition of some or all off the tissue at the poration site by creating a plasma at the poration site (e.g. laser);
heating via conductive materials;
high voltage AC current;
pulsed high voltage DC current;
micro abrasion using micro particles;
pressurised fluid (air, liquid);
pyrotechnic;
Electron beam or ion beam.

As used herein, “tissue” means any component of an organism including but not limited to, cells, biological membranes, bone, collagen, fluids and the like comprising some portion of the organism.

As used herein “puncture” or “micro-puncture” means the use of mechanical, hydraulic, sonic, electromagnetic, or thermal means to perforate wholly or partially a biological membrane such as the skin or mucosal layers of a human being or a mammal, or the outer tissue layers of a plant.

To the extent that “ablation” and “puncture” accomplish the same purpose of poration, i.e. the creating a hole or pore in the biological membrane optionally without significant damage to the underlying tissues, these terms may be used interchangeably.

As used herein “puncture surface” means the surface of the hole or pore at the outer surface of the biological membrane, which has been ablated or punctured.

As used herein the terms “transdermal” or “percutaneous” or “transmembrane” or “transmucosal” or “transbuccal” or “transtissual” or “intratissual” means passage of a permeant into or through the biological membrane or tissue to deliver permeants intended to affect subcutaneous layers and further tissues such as muscles, bones. In one embodiment the transdermal delivery introduces permeants into the blood, to achieve effective therapeutic blood levels of a drug.

As used herein the term “intradermal” means passage of a permeant into or through the biological membrane or tissue to delivery the permeant to the dermal layer, to therein achieve effective cosmetic tissue levels of a drug, or to store an amount of drug during a certain time in the biological membrane or tissue, for example to treat conditions of the dermal layers beneath the stratum corneum.

As used herein, “permeation surface” means the surface of the tissue surrounding the micropore or pore. “Permeation surface” may mean the surface of an individual micropore or pore, or may mean the total permeation surface, which means the sum of all individual surfaces of all individual micropores or pores.

As used herein, “corrected permeation surface” means the permeation surface corrected by a factor or a specific amount, for example by subtracting the surface of the micropore or pore which is part of the stratum corneum.

As used herein, the term “bioactive agent,” “permeant,” “drug,” or “pharmacologically active agent” or “deliverable substance” or any other similar term means any chemical or biological material or compound suitable for delivery through the biological membrane or tissue. This invention is not drawn to delivery of permeants. Rather it is directed to creating an initial permeation surface in a biological membrane like the skin.

As used herein, an “effective” amount of a permeant means a sufficient amount of a compound to provide the desired local or systemic effect.

As used herein, a “biological membrane” means a tissue material present within a living organism that separates one area of the organism from another and, in many instances, that separates the organism from its outer environment. Skin and mucous and buccal membranes are thus included as well as the outer layers of a plant. Also, the walls of a cell, organ, tooth, bone, or a blood vessel would be included within this definition.

As used herein, “transdermal flux rate” is the rate of passage of any bioactive agent, drug, pharmacologically active agent, dye, particle or pigment in and through the skin separating the organism from its outer environment. “Transmucosal flux rate” refers to such passage through any biological membrane.

The term “individual pore” as used in the context of the present application refers to a micropore or a pore, in general a pathway extending from the biological membrane. The biological membrane for example being the skin, the individual pore then extending from the surface of the skin through all or significant part of the stratum corneum. In the most preferred embodiment the pathway of the individual pore extending through all the stratum corneum and part of the epidermis but not extending into the dermis, so that no bleeding occurs. In the most preferred embodiment the individual pore having a depth between 10 μm (for newborns 5 μm) and 150 μm.

As used herein the term “initial microporation” refers to the total number of pores created. “Initial microporation dataset” refers to the set of data, wherein the initial microporation is defined. The dataset including at least one parameter selected from the group consisting of cross-section, depth, shape, permeation surface, total number of individual pores, geometrical arrangement of the pores on the biological membrane, minimal distance between the pores and total permeation surface of all individual pores. Preferably the initial microporation dataset defines the shape and geometrical arrangement of all individual pores, which then will be created using the microporator, so that the thereby created initial microporation is exactly defined and can be reproduced on various locations on the biological membrane, also on different objects, subjects or persons.

The plurality of laser pulses applied onto the same pore allows creating individual pores having a reproducible shape of the wall surrounding the individual pore and preferably allows also creating a reproducible shape of the lower end of the individual pore. The surface of the wall and the lower end is of importance, in particular the sum of the surface of the wall and the surface of the lower end which are part of the epidermis or the dermis, because this sum of surfaces forms a permeation surface through which most of the permeate passes into the tissue, for example into the epidermis and the dermis.

In a further embodiment the micro-porator is able to detect the depth at which the stratum corneum ends, e.g. the epidermis starts, or is able to detect the depth or thickness of the epidermis, for example, by using a spectrograph. This allows measuring the thickness of the stratum corneum and for example altering the total depth of created pores. With the initial microporation dataset, usually also the final depth of each individual pore is defined. This final depth can now be corrected in that the thickness of the stratum corneum is added. The individual pore is then created with this corrected depth, which means the individual pore becomes deeper, and which means that the permeation surface of the epidermis corresponds to the given permeation surface. This is of importance, because the transdermal flux rate, depending on the drug applied, often depends on the size of permeation surface which allows a high passage of drugs, which might be the permeation surface of the epidermis only.

If the depth of the individual pore is not corrected by the thickness of the stratum corneum, the effect of the stratum corneum can be considered by calculating a corrected permeation surface. This corrected permeation surface for example comprising only the permeation surface of the epidermis.

If the depth of the individual pore is not corrected by adding the thickness of the stratum corneum, for example, because this would lead to an individual micropore ending in the dermis, an additional micropore can be created, which comprises within the epidermis a surface corresponding at least to the surface of the stratum corneum.

The total permeation surface of all individual pores can also be determined. Knowing the corrected permeation surface, which means the permeation surface of the epidermis, allows one to better control or predict the transdermal delivery of drug into the patient, e.g. to better control or predict the release of the drug into the patient.

The micro-porator can create a microporation having a number of individual pores in the range between 10 and up to 1 million, and having individual pores with a width between 0.01 and 0.5 mm, and a depth between 5 μm and 200 μm or even more, as defined by the initial microporation dataset.

It can be advantageous for the application of specific permeants to create micropores, at least some micropores of a micro poration, to extend up to the dermis, so the specific permeant gets direct access to deep tissue layers.

In a preferred embodiment the micro-porator comprises an interface to at least read the initial microporation dataset, and to preferably read further parameters like permeant information, user information or porator application information. In a further preferred embodiment the micro-porator comprises a database that stores a plurality of initial microporation datasets. In a further preferred embodiment the micro-porator comprises a selector, which manually or automatically selects, for example based on user information such as the age, the most appropriate initial microporation dataset. The pores are then created according to this most appropriate initial microporation dataset.

The micro-porator according to the invention allows creating on a biological membrane a wide variety of different, reproducible microporations, such as a wide variety of initial permeation surfaces, and such as a wide variety of different decreases of the permeation surface over time. The permeation surface affects the transdermal or intradermal delivery of the permeant like the drug. Therefore even the same drug or the same amount of drug applied onto the skin can be delivered differently into the skin, depending on the permeation surface.

One advantage of the invention is that the puncture surface on the biological membrane is very small, which causes no damage of the biological membrane. The method according to the invention causes also no pain.

The micro-porator for porating a biological membrane may be designed, for example, as the laser micro-porator disclosed in PCT patent application No. PCT/EP05/XXXXX of the same applicant, filed on the same day and entitled “Laser microporator and method for operating a laser microporator”. The micro-porator for porating a biological membrane may comprise or being part of an integrated drug administering system, for example, as the system disclosed in PCT patent application No. PCT/EP2005/051702 of the same applicant, filed on the same day and entitled “Microporator for porating a biological membrane and integrated permeant administering system”. All citations herein are incorporated by reference in their entirety.

### BRIEF DESCRIPTION OF THE DRAWINGS

The present invention can be better understood and its advantages appreciated by those skilled in the art by referencing to the accompanying drawings, which are incorporated herein by reference. Although the drawings illustrate certain details of certain embodiments, the invention disclosed herein is not limited to only the embodiments so illustrated. Unless otherwise apparent form the context, all ranges include the endpoints thereof.

FIG. 1 shows a schematic cross-section of one pore of a laser porated skin;

FIG. 1a shows a schematic cross-section of three pores of a laser porated skin

FIG. 2 shows a laser micro-porator device;

FIG. 2a, 2b show a parallel or quasi-parallel laser beam;

FIG. 2e shows a lateral view of a pore;

FIG. 2c, 2d show a lateral view of further pores;

FIG. 3a-3c are perspective view of examples of suitable shapes of microporations;

FIG. 3d, 3f shows a plan view of the skin with an array of micro-porations;

FIG. 3e shows a schematic cross-section of a porated skin with a drug container attached to the skin surface;

FIG. 4a-4b shows the permeation surface of all micropores over time;

FIG. 5 shows a given permeation surface and a created permeation surface over time;

FIG. 6 shows transdermal delivery of a drug over time, in combination with a permeation surface;

FIG. 7a-7b show the serum concentration of a drug over time, with the same amount of drug but different permeation surfaces.

### DETAILED DESCRIPTION

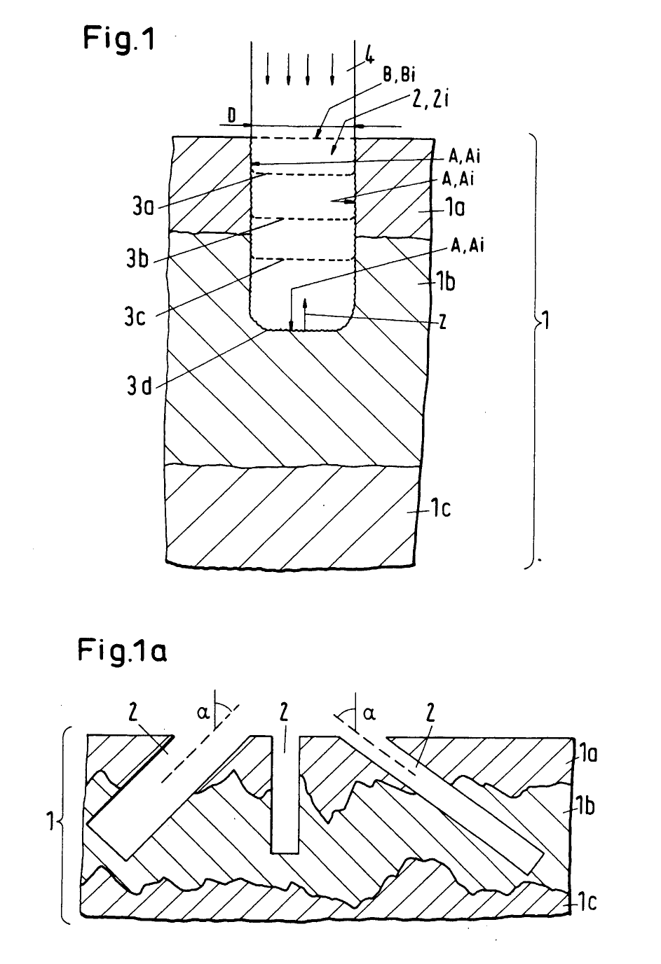

FIG. 1 shows a cross-sectional view of the top layers of the biological membrane 1, a human skin, including a stratum corneum 1a, an epidermal layer or epidermis 1b and a dermal layer or dermis 1c. The stratum corneum 1a is continuously renewed by shedding of corneum cells, with an average turnover time of 2-3 weeks. Underlying the stratum corneum 1a is the viable epidermis or epidermal layer 1b, which usually is between 50 and 150 μm thick. The epidermis contains no blood vessels and freely exchanges metabolites by diffusion to and from the dermis 1c, located immediately below the epidermis 1b. The dermis 1e is between 1 and 3 mm thick and contains blood vessels, lymphatics and nerves. Once a drug reaches the dermal layer, the drug will generally perfuse through system circulation.

FIG. 1 also shows a parallel or quasi-parallel laser beam 4 having a circular shape with a diameter D and acting on the surface of the skin 1. The impact of the laser beam 4 onto the skin 1 causes an ablation of the tissue. A first shot of the laser beam 4 causes an individual micropore 2 with a lower end 3a. The first shot effecting an individual puncture surface Bi at the outer surface of the skin 1 in the size of about (D/2)²*p, which corresponds to the amount of the outer surface of the biological membrane, which has been ablated or punctured. A second shot of the laser beam 4 at the same location causes an increase in depth of the individual pore 2 up to the lower end 3b, and a third and forth shot at the same location causes a further increase in depth up to the lower ends 3c and 3d. The total surface of the tissue 1 surrounding the individual pore 2 corresponds to the individual permeation surface Ai of the respective individual micropore 2. There is no tissue 1 at the individual puncture surface Bi, therefore the puncture surface Bi is not part of the individual permeation surface Ai.

The method according to the invention creates an initial permeation surface A in the biological membrane 1, the method comprising creating a plurality of individual micropores 2i in the biological membrane 1, each individual micropore 2i having an individual permeation surface Ai, the initial permeation surface A being the sum of the individual permeation surfaces Ai of all individual micropores 2i, after terminating the poration. Preferably such a number of individual micropores 2i and of such shapes is created, that the initial permeation surface A has a desired, predetermined value.

The total puncture surface B is the sum of all individual puncture surfaces Bi of all individual micropores 2i. In an advantageous method the individual micropores 2i are created with such a shape, that the initial permeation surface A is between 2 and 10 times bigger than the total puncture surface B.

Depending manly on properties of the tissue and the energy density of the pulsed laser beam 4, the increase in depth per pulse varies. Even though also a focused laser beam 4 might be used, the use of a non-focused laser beam 4 with a parallel or quasi-parallel laser beam 4 has the advantage, as disclosed in FIG. 1, that the individual permeation surface Ai of the individual pore 2i usually has a precise shape, for example a cylindrical shape. In the most preferred embodiment, the laser beam 4 is actuated such that the lower end 3c of the individual pore 2i is somewhere within the epidermis 1b but doesn't reach the dermis 1c.

Each individual pore 2 of the epidermis has a cell growth of usually (untreated) 3 to 15 μm per day, the cells usually growing from the lower end of the individual pore 2 in direction Z to the stratum corneum 1a. Which means the lower end 3d of the individual pore 2 is moving into the direction of the stratum corneum with a speed of about 3 to 15 μm/day, thereby reducing the permeation surface A. The corrected permeation surface, being the permeation surface of the epidermis only, without the surface of the stratum corneum, becomes the size of the puncture surface, which means the surface of the hole in the stratum corneum, as soon as the cells have reached the stratum corneum 1a. The remaining hole in the stratum corneum will by the time be filed by death cells of the epidermis, which significantly increases the barrier properties in the remaining hole, and which regenerates the stratum corneum. At the end the individual pore 2 has vanished due to cell growth, and the formerly ablated tissue is regenerated by new cells. The individual permeation surface Ai, as shown in FIG. 1, becomes zero when the cell reach the skin surface, which means that the whole individual pore 2i is filed with cells.

FIG. 1a shows three pores 2. The pore 2 in the middle is perpendicular with respect to the surface of the skin 1, whereas the pores 2 to the left and right penetrate with an angle a into the skin 1, the angle a being in a range between 0° and up to 70°. The advantage of this arrangement of the pore 2 is that the total length of the pore 2 can be very long, without the pore 2 entering into the dermis 1c. The pore 2 to the left or right can for example have double the length of the pore 2 in the middle, including a bigger permeation surface A.

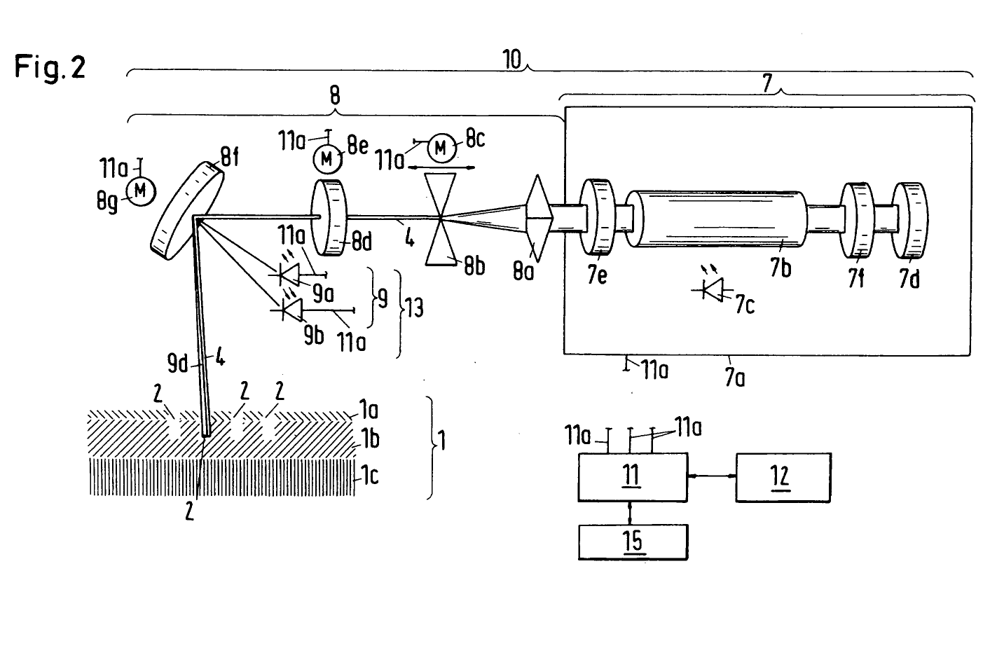

FIG. 2 shows a laser micro-porator 10 comprising a laser source 7 and a laser beam shaping and guiding device 8. The laser source 7 comprises a laser pump cavity 7a containing a laser rod 7b, preferably Er doped YAG, an exciter 7c that excites the laser rod 7b, an optical resonator comprised of a high reflectance mirror 7d positioned posterior to the laser rod and an output coupling mirror 7e positioned anterior to the laser rod, and an absorber 7f positioned posterior to the laser rod. The diverging lens 8b can be moved by a motor 8c in the indicated direction. This allows a broadening or narrowing of the laser beam 4, which allows changing the width of the laser beam 4 and the energy fluence of the laser beam 4. A variable absorber 8d, driven by a motor 8e, is positioned beyond the diverging lens 8b, to vary the energy fluence of the laser beam 4. A deflector 8f, a mirror, driven by an x-y-drive 8g, is positioned beyond the absorber 8d for directing the laser beam 4 in various directions, to create individual pores 2 on the skin 1 on different positions. A control device 11 is connected by wires 11a with the laser source 7, drive elements 8c, 8e, 8g, sensors and other elements not disclosed in detail.

In a preferred embodiment the laser porator 10 also includes a feedback loop 13. In FIG. 2, the feedback loop 13 comprises an apparatus 9 to measure the depth of the individual pore 2, and preferably includes a sender 9a with optics that produce a laser beam 9d, and a receiver with optics 9b. The laser beam 9d has a smaller width than the diameter of the individual pore 2, for example five times smaller, so that the laser beam 9d can reach the lower end of the individual pore 2. The deflection mirror 8f directs the beam of the sender 9a to the individual pore 2 to be measured, and guides the reflected beam 9d back to the receiver 9b. In a preferred embodiment, the depth of the individual pore 2 is measured each time after a pulsed laser beam 4 has been emitted to the individual pore 2, allowing controlling the effect of each laser pulse onto the depth of the individual pore 2. The apparatus 9 may be able to detect further characteristics of the individual micropore 2i, like depth, diameter, cross section or shape or surface. The feedback loop 13 may, for example, comprise a sender 9a and a receiver 9b, built as a spectrograph 14, to detect changes in the spectrum of the light reflected by the lower end of the individual pore 2. This allows, for example, detecting whether the actual lower end 3a, 3b, 3e, 3d of the individual pore 2 is part of the stratum corneum 1a or of the epidermis 1b. This also allows measuring the thickness of the stratum corneum 1a. The laser porator 10 also comprises a poration memory 12 containing specific data of the individual pores 2, in particular the initial microporation dataset. The laser porator 10 preferably creates the individual pores 2 as predescribed in the poration memory 12. The laser porator 10 also comprises one or more input-output device 15 or interfaces 15, to enable data exchange with the porator 10, in particular to enable the transfer of the parameters of the individual pores 2, the initial microporation dataset, into the poration memory 12, or to get data such as the actual depth or the total surface Ai of a specific individual pore 2i.

The pulse repetition frequency of the laser source 7 is within a range of 1 Hz to 1 MHz, preferably within 100 Hz to 100 kHz, and most preferred within 500 Hz to 10 kHz. Within one application of the laser porator 10, between 2 and 1 million individual pores 2 can be produced in the biological membrane 1, preferably 2 to 10000 individual pores 2, and most preferred 10 to 1000 individual pores 2, each pore 2 having a width or diameter in the range between 0.001 mm and 0.5 mm, and each pore 2 having a depth in the range between 5 μm and maximal 250 μm, the lower end of the individual pore 2 preferably being within the epidermis 1b. If necessary, the porator is also able to create pores 2 with a depth of more than 250 μm.

FIG. 2 discloses a circular laser beam 4 creating a cylindrical individual pore 2. The individual pore 2 can have other shapes, for example in that the laser beam 4 has not a circular but an elliptical shape. The individual pore 2 can also be shaped by an appropriate movement of the deflector 8f, which allows creation of individual pores 2 with a wide variety of shapes.

In a preferred embodiment the feedback loop 9, 13 is operatively coupled to the poration controller 11, which, for example, can compare the depth of the individual pore 2 with a predetermined value, so that no further pulse of the laser beam 4 is directed to the individual pore 2 if the characteristic of the individual pore 2, for example, the depth, is greater than or equal to a preset value, or if the characteristic of the individual pore 2 is within a preset range. This allows creation of individual pores 2 with a predetermined depth as well as a predetermined individual permeation surface Ai.

FIGS. 2a and 2b disclose a laser beam 4a, herein referred to as a parallel or quasi-parallel laser beam. The laser beam 4a has a propagation direction vector vpd of the laser beam 4a and a divergence vector vd of the main divergence of the laser beam 4a. The angle β between the direction vector vpd and the divergence vector vd is less than 3°, preferably less than 1° and most preferred less than 0.5°. This means the parallel or quasi-parallel laser beam 4a has a divergence of less than 3°. The diameter of the parallel or quasi-parallel laser beam 4a can become wider as it propagates in vector direction vpd, as disclosed in FIG. 2a, or can become narrower, as disclosed in FIG. 2b. The parallel or quasi-parallel laser beam 4a shows the properties disclosed in FIGS. 2a and 2b at least within a certain range of focus, the focus or focus range, extending in direction of the propagation direction vector vpd, has a range of about 1 cm to 5 cm, preferably a range of 2 cm to 3 cm.

FIG. 2e shows a schematic representation of the lateral view of a pore 2 produced in the skin 1 by the laser beam 4a. The laser beam 4a having a homogeneous energy density, which can be reached by the use of optics, e.g. Gaussian lens, or by a multimode laser beam generation. The laser beam 4a has a so called top hat profile. The laser beam 4a is almost homogeneous with respect to divergence and energy distribution. This laser beam 4a therefore causes a defined ablation of the skin 1 regarding depth and shape. In contrast a laser beam 4 without a homogeneous energy density and/or a laser without a parallel or quasi-parallel laser beam 4 may cause a pore 2 in the skin 1 as disclosed in FIGS. 2c and 2d. Such a laser beam 4 may create pores 2 which damage the sensitive layer between the epidermis and the dermis, so that bleeding and pain occurs. The laser beam 4a as disclosed in FIG. 2e has the advantage that the effect of energizing or heating of adjacent tissue is very low, which causes less destruction of cells. A further advantage is that the shape of the pore 2 from top to bottom is kept the same, so that a very exact and reproducible pore 2 is generated. A further advantage is that the measurement of the depth of the pore 2 is easy and precise, because the bottom end of the pore 2 can easily be detected. In contrast the pores 2 disclosed in FIGS. 2c and 2d have no clear bottom end. Therefore it is more difficult or even not possible to measure the depth of these pores 2 and to calculate its permeation surface.

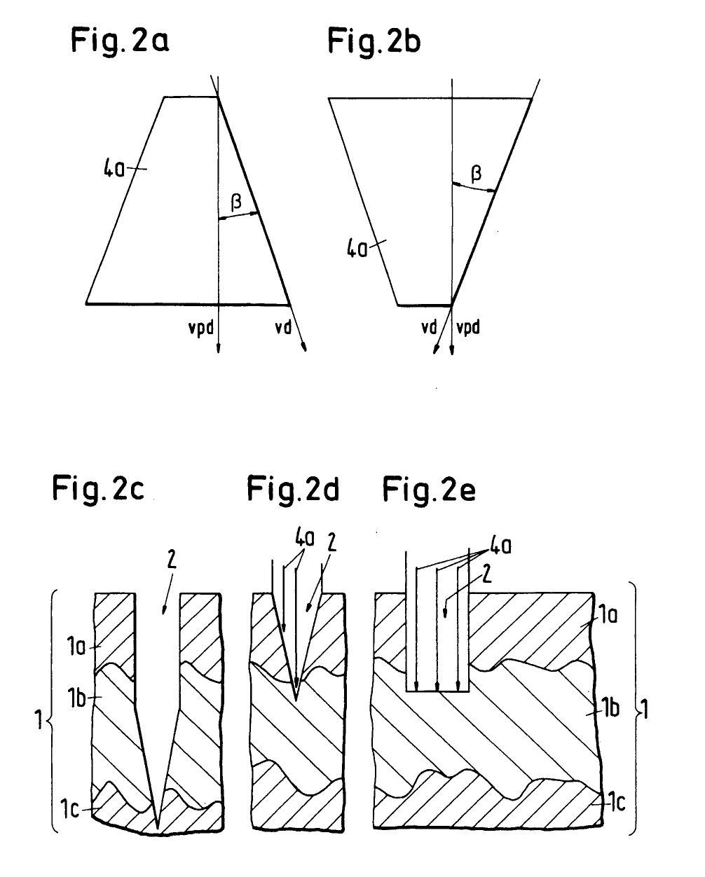

FIG. 3a shows an array of individual pores 2 in the skin 1. All individual pores 2 have the same shape and depth.

FIG. 3b shows examples of individual pores 2a to 2f of various shapes, which can be created with the laser porator 10. To produce the individual pores shown in FIG. 3b, at least the cross-section of the laser beam 4 has to be varied. In a preferred embodiment, the laser porator 10 varies the cross-section and/or the energy density of each consecutive pulsed laser beam 4, which allows creation of individual pores 2 with numerous different shapes. If the ablated layer per laser beam pulse 4 is very small, even conically shaped individual pores 2g, 2h, 2i, as disclosed in FIG. 3c, can be created.

FIG. 3d shows a plan view of the skin having a regular array of individual pores 2 that collectively form a micro-poration. The micro-poration on the biological membrane, after the laser porator 10 has finished porating, is called “initial microporation”. The poration memory 12 contains the initial microporation dataset, which define the initial microporation. The initial microporation dataset comprises any suitable parameters, including: width, depth and shape of each pore, total number of individual pores 2, geometrical arrangement of the pores 2 on the biological membrane, minimal distance between the pores 2, and so forth. The laser porator 10 creates the pores 2 as defined by the initial microporation dataset. This also allows arranging the individual pores 2 in various shapes on the skin 1, as for example disclosed with FIG. 3f.

FIG. 3e discloses a patch 5 comprising a container 5a with a drug or cosmetic substance and an attachment 5b, which is attached onto the skin 1, the container 5a being positioned above an area comprising individual pores 2. The area can have a surface, depending on the number and spacing of the individual pores 2, in the range between 0.1 mm² and 1600 mm², preferred between 1 mm² and 200 cm², and also preferred 20×20 mm, e.g. a surface of 400 mm².

For each individual pore 2i, the surface of the inner wall and the surface of the lower end are of importance, in particular the individual permeation surface Ai, being the sum of both of these surfaces. In a preferred embodiment, the laser porator 10 comprises the distance measurement apparatus 9, which facilitates determining the individual permeation surface Ai very accurately. The individual permeation surface Ai can easily be calculated for each individual pore 2i. If the individual pore 2i has the shape of, for example, a cylinder, the individual permeation surface Ai corresponds to the sum of D*p*H and (D/2)²*p, D being the diameter of the individual pore 2, and H being the total depth of the individual pore 2. The effective individual permeation surface Ai of the individual pore 2i often doesn't correspond exactly to the geometrical shape defined by D and H, because the surface of the individual pore 2i may be rough or may comprise artefacts, which means the effective permeation surface is bigger than the calculated individual permeation surface Ai. The individual permeation surface Ai is at least a reasonable estimate of the effective permeation surface. Usually there, is only a small or no difference between the individual permeation surface Ai and the effective permeation surface in the individual pore 2i. The total permeation surface A of n individual pores 2i is then the sum of all individual permeation surfaces Ai of all n individual pores 2i.

In a further embodiment, the thickness of the stratum corneum, or if necessary also the beginning of the dermis, which means the thickness of the stratum corneum plus the thickness of the epidermis, can be measured. This in turn permits to calculate a corrected permeation surface Ai for each individual pore 2i, by subtracting the permeation surface of the stratum corneum from the individual permeation surface Ai, which establishes the effective permeation surface of the epidermis 1b. On the other hand, the depth of the individual pore 2i can be increased by the thickness of the stratum corneum, so that the given individual permeation surface Ai corresponds to the permeation surface of the epidermis 1b. If this increase in depth should result in an individual pore 2i extending to within the dermis, the depth of the individual pore 2i will not be increased, but an additional micropore created, comprising a surface within the epidermis which compensates the surface of the former individual micropore 2i, which is part of the stratum corneum.

Each individual pore 2 of the epidermis has a cell growth of usually 3 to 15 μm per day, the cells growing from the lower end of the individual pore 2 in direction Z to the stratum corneum 1a. This cell growth causes the individual permeation surface Ai of each individual pore 2i respectively the total permeation surface A of all individual pores 2 to decrease in function of time. Depending on the total number of individual pores 2, which can be in a range of up to 100 or 1000 or 10000 or even more, the geometrical shape of the individual pores 2, and taking into account the effect of cell growth, the total permeation surface in function of time can be varied in a wide range.

The initial permeation surface and also the decrease of the permeation surface over time can be predicted and calculated by an appropriate choice of the number of pores 2 and their geometrical shape. The method according to the invention therefore comprises: evaluating the decrease of the individual permeation surface Ai of the individual micropore 2i due to cell growth; evaluating the total permeation surface over time A(t), which is the sum of the individual permeation surfaces Ai, and selecting an appropriate number and an appropriate shape of individual micropores 2i so that the total permeation surface over time A(t) corresponds to a given permeation surface over time. This definition of number and shape of all pores is stored as the initial microporation dataset D. Correction factors may be applied to this initial microporation dataset D, for example taking into account the thickness of the stratum corneum, or based on user information like individual speed of cell growth, or based on the optional use of regeneration delayer like occlusive bandage, diverse chemical substances, etc., which influence the speed of cell growth.

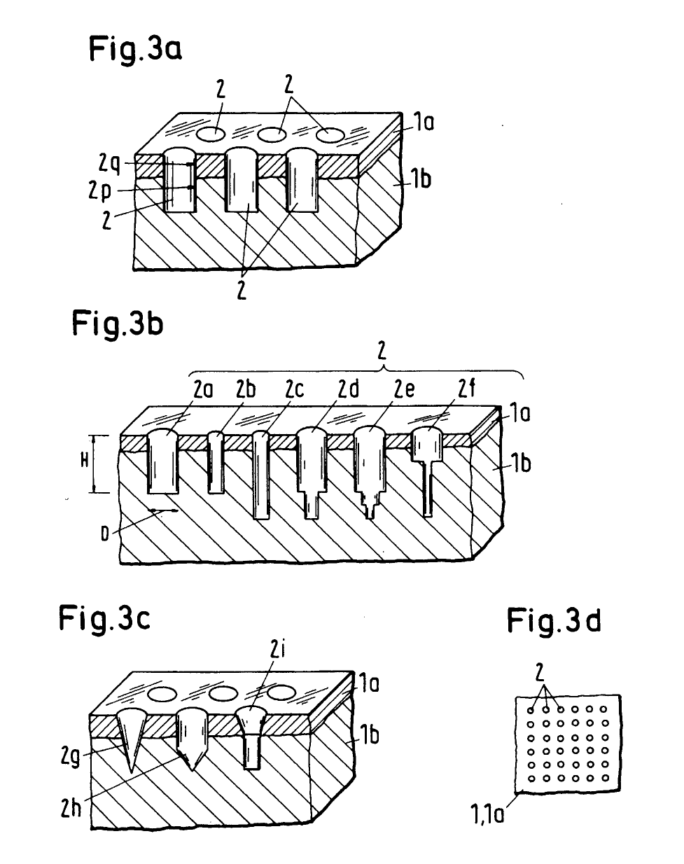

FIGS. 4a and 4b show examples of the total permeation surface A(t) over time. FIGS. 4a and 4b show the corrected total permeation surface A(t), which is the total permeation surface A(t) of the epidermis 1a only. The laser-porator 10 allows to micro-porating a biological membrane 1 by the creation of an array of micropores 2 in the biological membrane 1, whereby the number of micropores 2 and the shape of these micropores 2 is created according to the given initial microporation dataset D, so that an initial permeation surface A is created, and so that permeation surface decreases, due to cell growth, over time, as defined by the total permeation surface over time A (t).

In a preferred method the microporation consists of a plurality of different groups of micropores 2i, all micropores 2i of the same group having the same shape and size. For example the Initial microporation dataset D according to FIG. 4a comprises three groups of cylindrical micropores 2, all micropores 2 of the same group having the same shape:
a first group consisting of 415 pores with a diameter of 250 μm, a depth of 50 μm and a permeation surface A1 as a function of time.
a second group consisting of 270 pores with a diameter of 250 μm, a depth of 100 μm and a permeation surface A2 as a function of time.
a third group consisting of 200 pores with a diameter of 250 μm a depth of 150 μm and a permeation surface A3 as a function of time.

The total permeation surface A (t) as a function of time is the sum of all three permeation surfaces A1, A2 and A3.

All individual pores 2i, which means the initial microporation, is created within a very short period of time, for example, within up to one second, so that beginning with the time of poration TP, the sum of all created pores 2i forming an initial permeation surface, which, due to cell growth, decreases as a function of time. At the time TC all individual pores 2i are closed, which means that the value of the total permeation surface A (t) becomes very small or zero.

The initial microporation dataset according to FIG. 4b consists also in three groups of cylindrical micropores 2:
a first group consisting of 4500 pores with a diameter of 50 μm, a depth of 50 μm and a permeation surface A1 as a function of time.
a second group consisting of 2060 pores with a diameter of 50 μm, a depth of 100 μm and a permeation surface A2 as a function of time.
a third group consisting of 1340 pores with a diameter of 50 μm, a depth of 150 μm and a permeation surface A3 as a function of time.

The total permeation surface A is the sum of all three permeation surfaces A1, A2 and A3.

Depending on the number of pores 2 and their shape, in particular the diameter and depth of the pores 2, the total permeation surface A(t) over time can be varied and adopted in a wide range. This makes it clear that the poration of individual pores 2 does not only determine the initial permeation surface, but also the function of the total permeation surface A (t) over time. FIGS. 4a and 4b show the total permeation surface A(t) over a time period of 9 days, starting with an initial permeation surface of 90 mm². The total permeation surface A (t) decreases within 9 days to a very small value or to zero. Depending on the shape of the individual pores 2, the time period may be much shorter, for example, just 1 day, or even shorter, for example, a view hours.

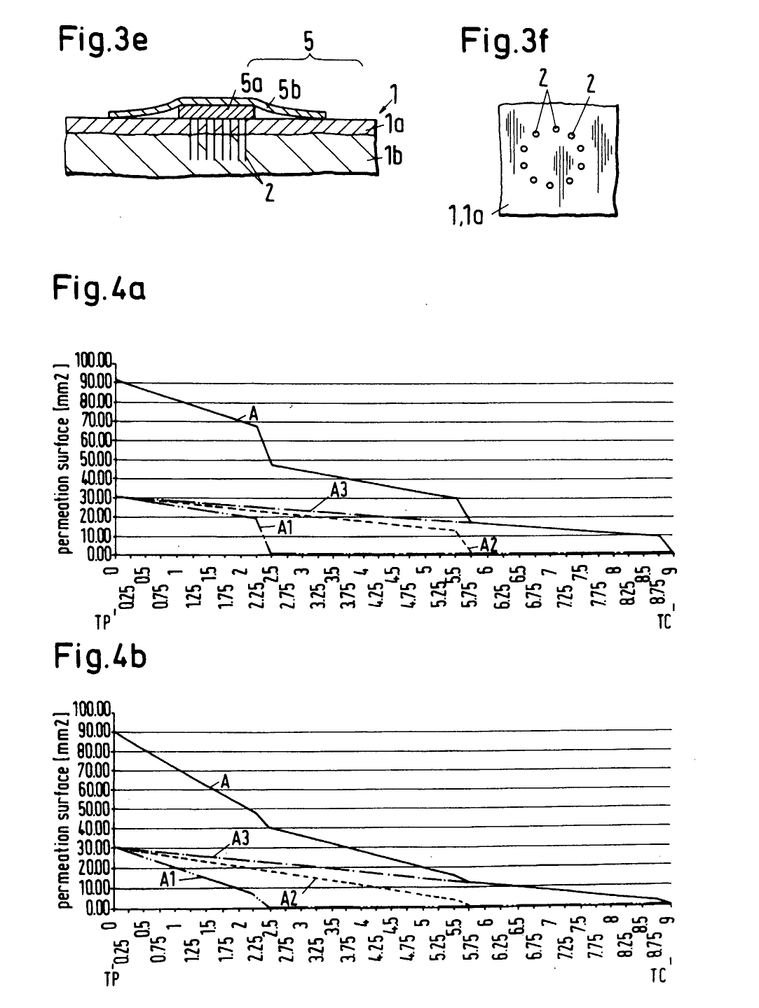

Almost any total permeation surface A(t) as a function of time may be establish by a proper selection of the number and the shape of the individual pores 2. FIG. 5 shows a given function A_{G} of a permeation surface as a function of time. FIG. 5 also shows the permeation surface over time of different groups A1, A2, A3, A4, A5, . . . of individual micropores 2i having the same shape. Each group being defined by the number of pores, the diameter and the depth. AU individual pores 2 have cylindrical shape. By combining the individual permeation surfaces (A1, A2, A3, A4, A5, . . . ) of all the groups, a total permeation surface A(t) over time is achieved, which function is quite similar to the given function A_{G}. The different groups of individual pores, their number and their shape can be determined by mathematical methods known to those skilled in the art.

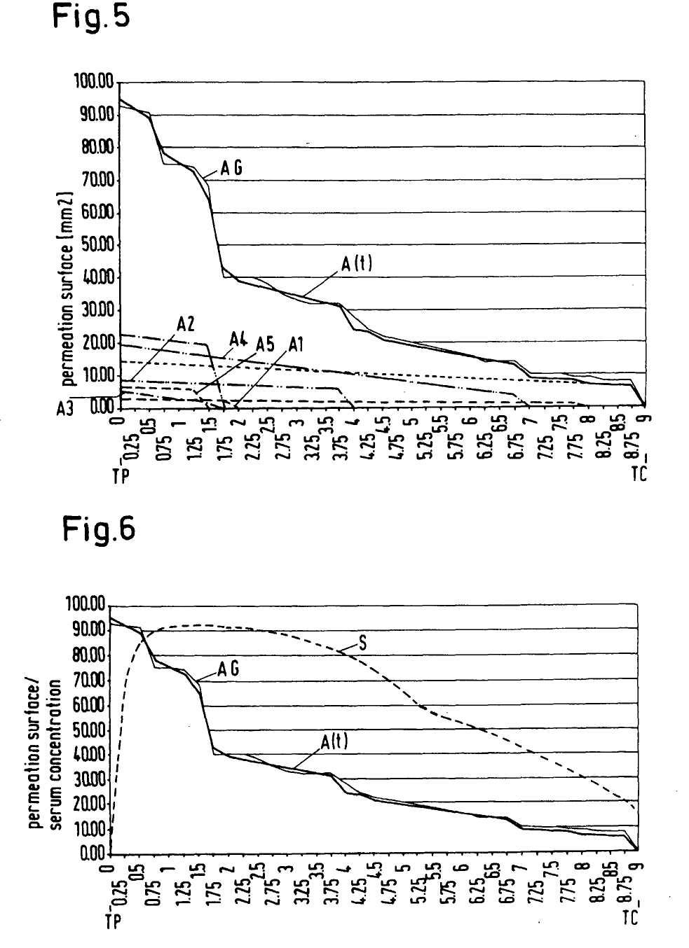

FIG. 3e shows a patch 5 containing a drug 5a and being fixed onto the skin 1, above the individual pores 2. FIG. 6 shows the serum concentration S of this drug as a function of time in the blood. The drug is entering the permeation surface by passive diffusion. The amount of drug entering the permeation surface is mainly determined by the total permeation surface A(t) over time. Therefore, the serum concentration as a function of time is influenced by an appropriate poration of the skin 1 with an initial microporation, before the drug is applied onto the skin. This also makes it clear that the method for creating a permeation surface in a biological membrane is finished before the drug is applied. Therefore this method is completely independent from applying a permeant like a drug.

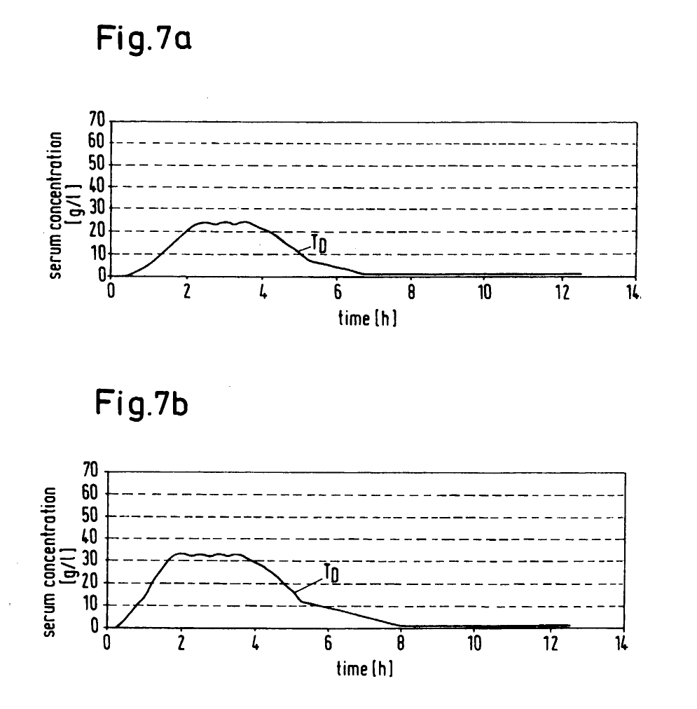

FIG. 7a to 7b show the administration of the same amount of drug, for example 100 mg acetylsalicylic acid, the drug being arranged on the skin 1 as disclosed in FIG. 3e and the skin 1 being microporated with two different initial poration datasets D, causing two different total permeation surfaces A(t) over time. Combining properties of the drug and depending on the appropriate choice of a total permeation surface A(t) as a function of time, the level of the serum concentration as well as the time period within which the drug is released, can be predescribed. The total permeation surfaces over time A(t) are not disclosed in the figures, but their effect on the level of serum concentration. In FIG. 7a the total permeation surface A(t) is chosen such, in combination with the drug, that the maximal serum concentration is about 25 g/l over a short period of time of about two hours. FIG. 7b shows the effect of another total permeation surface A(t), which causes a fast application (turbo) of the drug, with maximal serum concentration of about 30 g/l over a short period of time of about two hours. Such short periods of application time may be achieved by creating an appropriate total permeation surface A(t) in the epidermis 1b, which surface decreases very fast after for example 4 hours. This can for example be achieved by a microporation comprising individual pores 21 having lower ends 3d at the border between the stratum corneum 1a and the epidermis 1b. As can be seen in FIG. 1, the corrected individual permeation surface of such an individual pore 2i, which means the permeation surface of the epidermis 1b, corresponds to the puncture surface Bi. Because this permeation surface is just at the transition area between epidermis 1b and stratum corneum 1a, this permeation surface will, due to cell growth, decrease very fast over time, thereby reducing the transdermal flux rate very fast. If, for example, a very high transdermal flux rate is required at the beginning, over a short period of time, this can be achieved by creating a lot of micropores having their individual permeation surfaces in the epidermis 1b, but the lower end 3d of the individual pores 2i being very close to or at the border between the stratum corneum 1a and the epidermis 1b. For example a group of 50 to 1000 individual micropores 2i, having a diameter of 500 μm and a depth corresponding to the thickness of the stratum corneum could be created, just to get a large permeation surface during a short period of time. The individual permeation surfaces of these individual pores 2i will, due to cell growth, decrease fast over time.

An advantage is that the same amount of drug, e.g. the same patch, applied onto the skin 1, causes a different serum concentration, depending only on the function of the total permeation surface A over time. This allows administering the same drug in different ways. This also allows administering the same drug in an individual way, in that the total permeation surface A(t) over time is created depending on individual parameters of the person the drug is applied to.

The method for creating an initial permeation surface in a biological membrane can also as such be used for pure cosmetic treatment, in that the biological membrane 1, for example the skin, is porated with a plurality of individual pores 2. These pores 2 initiate a cell growth in the epidermis so that these pores 2, after a certain time, become filled with newly generated cells. The only object is to beautify the human or animal skin for cosmetic reasons. This cosmetic treatment, creating an array of micropores, can be repeated several times, for example every ten days, to cause a cell growth in a lot of different areas. Because the individual puncture surfaces Bi as well as the total puncture surface B are so small, this cosmetic treatment is not visible and does not damage the skin.

In this detailed description the creation of micropores 2 was, by way of example, described using a pulsed laser beam. It is apparent that other methods could also be suitable, based for example on mechanical, hydraulic, sonic, electromagnetic, electric or thermal energy. The micropores do also not necessarily need the shape of a hole, but may also have other shapes, for example, the shape a tunnel with two openings. The microporator should be able to reproducibly create micropores, and/or the microporator should comprise an apparatus 9 to measure characteristics of the individual micropores, so that a microporation with a predetermined initial poration, preferably a predetermined initial permeation surface may be created in a biological membrane.

1. A method for creating an initial permeation surface (A) in a biological membrane (1), the method comprising:
a) creating a plurality of individual micropores (2i) in the biological membrane (1), each individual micropore (2i) having an individual permeation surface (Ai); and
b) creating such a number of individual micropores (2i) and of such shapes, that the initial permeation surface (A), which is the sum of the individual permeation surfaces (Ai) of all individual micropores (2i), has a desired value.

2. The method of claim 1, wherein the desired value of the initial permeation surface (A) is between 2 mm² and 1000 mm².

3. The method of claim 1, further comprising detecting a characteristic of a selected one of the plurality of individual micropore (2i), the characteristic including at least one of: depth, diameter, cross section, shape, surface, and kind of tissue.

4. The method of claim 3, further comprising detecting the characteristic of the individual micropore (2i) at least twice during creation of the individual micropore (2i).

5. The method of claim 1, further comprising:
c) evaluating decrease of the individual permeation surface (Ai) of the individual micropore (2i) due to cell growth;
d) evaluating total permeation surface over time (A(t)), which is the sum of the individual permeation surfaces (Ai), and
e) selecting an appropriate number and an appropriate shape of individual micropores (2i) so that the total permeation surface over time (A(t)) corresponds to a given permeation surface over time.

6. The method of claim 1, further comprising creating at least 10 micropores (2).

7. The method of claim 1 wherein the micropores (2i) have the same shape.

8. The method of claim 1, wherein at least some of the plurality of micropores are distributed to form a plurality of different groups, in which all micropores (2i) of the same group have having the same shape and size.

9. The method of claim 1, wherein the step of creating each individual micropore (2i) ablates at the outer surface of the biological membrane (1) an individual puncture surface (Bi), and wherein the sum of puncture surfaces (Bi) of all micropores (2) corresponds to a total puncture surface (B).

10. The method of claim 9, comprising creating the micropores (2) with such a shape that the initial permeation surface (A) is between 2 and 10 times bigger than the total puncture surface (B).

11. The method of claim 1, comprising creating the plurality of micropores (2) with a diameter between 1 μm and 500 μm.

12. The method of claim 1, comprising creating the plurality of micropores (2) with a depth between 5 μm and 200 μm.

13. The method of claim 1, comprising creating the plurality of micropores (2) having a lower end within the epidermis.

14. The method of claim 1, comprising creating a group of micropores (2) having a lower end close to or at the transition of stratum corneum (1a) and epidermis (1b).

15. The method of claim 1, comprising creating the plurality of micropores (2) by the of mechanical, hydraulic, sonic, electromagnetic, or thermal energy.

16. The method of claim 1, comprising creating the plurality of micropores (2) by a pulsed laser beam.

17. The method of claim 1, wherein the plurality of micropores (2) provide an initial permeation surface (A) that becomes zero within a time range of 1 hour to 10 days.

18. The method of claim 1, further comprising detecting a thickness of the stratum corneum.

19. The method of claim 18, further comprising increasing a depth of the individual micropore (2i) by a respective thickness of the stratum corneum.

20. The method of claim 18, further subtracting a surface of the individual micropore (2i), which is part of the stratum corneum, from the individual permeation surface (Ai).

21. The method of claim 18, further creating an additional micropore (2i) comprising a surface within the epidermis which compensates for the surface of the individual micropores (2i), which is part of the stratum corneum.

22. Use of the method of claim 1 as a cosmetic method for the stimulation of cell growth in a biological membrane (1).

23. A method for administering a cosmetic substance, the method comprising:
e) creating a microporation in skin (1) according to the method of claim 1;
f) applying the cosmetic substance to the microporation such that the cosmetic substance is absorbed into the skin through micropores (2) of the microporation; and
g) wherein intradermal delivery of the cosmetic substance is a function of the initial permeation surface (A).

24. The method of claim 23, further comprising determining a total permeation surface over time (A(t)) to determine a flux rate of the cosmetic substance into the skin.

25. A Microporator (10) configured to allow operation according to a method of claim 1.

26. A Microporation created according to a method of claim 1, and comprising an initial permeation surface (A) of predetermined size.

27. The microporation of claim 26, comprising a predetermined total permeation surface over time (A(t)).

28. A method for administering a drug, the method comprising:
e) creating a microporation in a biological membrane (1) according to a method of claim 1;
f) applying the drug to the microporation such that the drug is delivered into the biological membrane through a plurality of micropores (2); and
g) wherein the delivery of the drug is determined by the initial permeation surface (A).

29. A method of claim 28, further comprising determining a total permeation surface over time (A(t)) which determines the delivery of the drug into the biological membrane.
